(19) United States
(12) Patent Application Publication
(10) Pub. No.: US 2002/0168418 A1
(43) Pub. Date: Nov. 14, 2002

(54) Method and apparatus for treating water for use in improving the intestinal flora of livestock and poultry

(75) Inventors: John d?apos;Arc Lorenz II, New York, NY (US); Scott A. Schorzman, Kenmore, WA (US); Troy T. Johnson, Portland, OR (US); John J. Orolin, West Linn, OR (US)

Correspondence Address: SEED INTELLECTUAL PROPERTY LAW GROUP PLLC, 701 FIFTH AVE, SUITE 6300, SEATTLE, WA, 98104-7092, US

(73) Assignee: H20 Technologies, Ltd., Milwaukie, OR

(21) Appl. No.: 10/072,651

(22) Filed: Feb. 6, 2002

Related U.S. Application Data
(63) Continuation-in-part of application No. 09/575,727, filed on Aug. 4, 2000.

(51) Int. Cl. A61K 33/00

(52) U.S. Cl. 424/600

(57) ABSTRACT

A water treatment system for treating water for use in improving the intestinal flora of livestock and poultry. The treated water for livestock and poultry use provides water with increased dissolved oxygen such that when the treated water is ingested, the livestock and poultry have increased lactic acid producing bacteria and decreased coliforms in the intestine. Increasing the molecular oxygen content in the intestine through providing the birds with treated water containing higher an increased level of dissolved oxygen alters the balance of flora in favor of the beneficial bacteria, thereby improving bird health and performance. By reducing the numbers of strict anaerobes in the gut of the growing bird, the risk of infectious disease, and hence morbidity and mortality are reduced. This allows the beneficial bacteria to proliferate thereby enhancing the digestion and absorption of available nutrients to the bird. The net effect of encouraging the beneficial bacteria, such as Lactobacilli, and suppressing the pathogenic bacteria such as Salmonella, Shigella, Staphylococcus, Escherichia coli, Clostridium and Helicobacter pylori, is greater body weight and improved feed efficiency and healthier animals with fewer antibiotics. The system includes a water treatment filter, a flow meter that coordinates with a flow switch and an electrocatalytic cell coupled to a holding chamber that is attached to an outlet of the cell.

### BACKGROUND OF THE INVENTION

1. Field of the Invention

This invention relates generally to a water treatment system and more particularly, to a method and apparatus for treating water for livestock and poultry use such that the livestock and poultry have increased lactic acid producing bacteria and decreased coliforms in the intestine when ingesting the treated water.

2. Description of the Related Art

The need for high quality water in livestock and poultry production is becoming increasingly essential. This is primarily due to the overall reduction in water quality and the trend towards larger and denser livestock and poultry populations. Water quality, whether it be ground or surface water, has been deteriorating over many years for reasons that range from animal waste and agricultural chemical runoff to lowered ground water tables. Occurrences of contamination from nitrates, bacteria, chemicals, iron, hydrogen sulfide, etc., have become more and more prevalent. Poor water quality has resulted in higher disease and morbidity rate in livestock and poultry, which has increased the need for antibiotic use.

Drinking water quality is an important factor for livestock and poultry health. Elevated concentrations of minerals, bacteria or toxic constituents in the water can have a detrimental effect on normal physiological processes in the body, thus causing inferior development, such as weight gain and growth. High concentrations of minerals can also restrict water flow to the birds by clogging the feeder lines. This can cause flooding of the drinkers and wet litter, which, in turn, can lead to disease and leg problems.

Within the digestive tract of the poultry, for example, there is a very diverse microbial population. A strong relationship exists between the bird and its microflora in terms of their influence over bird health and digestion/absorption of nutrients. The bacterial population within the gut is not stationary and may be subject to manipulation. This manipulation has traditionally been accomplished with antibiotic and other medicinal methods.

Within the gut, two groups of bacteria can be identified, namely those that can survive in the presence of oxygen (facultative anaerobes) and those that cannot (strict anaerobes). In general terms, strict anaerobes tend to be the more dominant group and may include harmful organisms such as E. coli, Salmonella and Clostridia. For these bacteria, molecular oxygen is a toxic substance that will either kill them or inhibit their growth. Facultative anaerobes present in the digestive tract of poultry, mainly Lactobacilli, are important due to them having an exclusively fermentative energy-yielding metabolism. Thus they prove beneficial to the bird in terms of aiding them to release more energy from their diet. Previously, bacteria growth was controlled in the digestive tract by antibiotic use. Antibiotics indiscriminately kill both groups of bacteria.

Various methods are being used to reduce the impurities that adversely effect water quality. Chlorination has been the most common method to treat water for bacterial contamination. Chlorination removes bacteria from the water supply by converting some of the chemical contaminants into less harmful forms. For example, chlorinization oxidizes nitrites to the less toxic nitrate form and reacts with hydrogen sulfide and ferrous iron to produce sulfates, ferris iron and other solid materials that can then be removed by filtration. Since chlorine reacts with organic compounds, however, its effectiveness as an antimicrobial agent is more quickly reduced if high levels of organic matter are present. Furthermore, although chlorination can kill some bacteria in the water supply, it does nothing to increase or improve the overall water quality of poor water.

Another method used to control the quality of water is with polyphosphates. Polyphosphates are chemical compounds used to prevent the build-up of scale in the water system by causing the minerals to go into solution. Yet another method for water treatment is magnetic devices that are designed to prevent scaling buildup in the water system.

Aeration equipment had been used to inject oxygen into water. The primary purpose of the process is to oxidize organic water in wastewater and potable water applications. Wastewater aeration is primarily done under atmospheric conditions for the purpose of aerobic digestion. Generally, in atmospheric applications, air is bubble diffused within a tank to accomplish oxidation. It might then be repressurized for distribution. A variety of ways are used to provide aeration under pressurized situations. Compressed air or concentrated oxygen could be injected into a water stream or could be drawn into a water stream with the aid of a venturi. In addition, water could be passed through an air pocket within a tank to accomplish aeration.

These conventional systems address specific problems with the quality of water, such as scaling, oxygen deficiency, bacterial contamination, etc., but do not provide an overall efficient system for treating and improving quality of water in a cost-efficient manner. If these systems fail, are not employed, or do not completely treat the water, antibiotics treat the diseases found in the livestock and poultry that was caused by contaminated water.

### BRIEF SUMMARY OF THE INVENTION

According to principles of the present invention, a water treatment system is provided for treating water for use with livestock and poultry. The water treatment system increases the dissolved oxygen in the water and precipitates out contaminants to produce a cleaner, less contaminated, higher quality of water in an efficient and cost effective manner. This treated water is used to suppress the harmful bacteria in the intestinal tract of the livestock or poultry and feed and proliferate the helpful bacteria in the intestinal tract of the livestock or poultry to produce a healthier animal without antibiotics.

The system includes a water treatment filter, a flow meter that coordinates with a flow switch and an electrocatalytic cell coupled to a holding chamber that is attached to an outlet of the cell. The water treatment filter removes materials from the water that alter the electrical properties of the water prior to the water entering the electrocatalytic cell. The electrocatalytic cell includes a plurality of conductive plates with spaces therebetween through which water may pass. An electric current flows across the conductive plates of the cell through the water, breaking some of the water molecules into their component parts of hydrogen gas and oxygen gas. At the outlet of the cell, both hydrogen gas and oxygen gas are present in the fluid.

The holding chamber is vertically oriented and longitudinally extending from the outlet of the electrocatalytic cell. The vertical length of the chamber is selected to provide sufficient time to allow a majority of the gaseous oxygen to transition to dissolved oxygen. A collection valve may be included at the top of the holding chamber to allow collection and release of accumulated gases. An optional sediment filter may be added after the outlet from the holding chamber prior to the water reaching the livestock drinking system. Additional features include bypass piping and valves allowing the water to flow around the system so that water flow to the animals is uninterrupted if maintenance is required on the system.

The system may further include the control unit allowing modification of the current density in the conductive plates of the electrocatalytic cell based on the value of the flow rate measured by the flow meter. The control unit may further provide a memory unit allowing recordation of information for a given amount of time. Access to the memory unit may be provided via a modem link, alternatively, a direct link at the control unit may also provide access to the memory unit.

Increasing the molecular oxygen content in the intestine through providing the birds with treated water containing higher an increased level of dissolved oxygen alters the balance of flora in favor of the beneficial bacteria, thereby improving bird health and performance. In understanding the effect of oxygenized water on the improvements in weight, feed efficiency and reduced mortality that have been observed under normal growing conditions, it is believed that this change to the gut microflora is significant.

By reducing the numbers of strict anaerobes in the gut of the growing bird, the risk of infectious disease, and hence morbidity and mortality are reduced. This in turn allows the beneficial bacteria to proliferate thereby enhancing the digestion and absorption of available nutrients to the bird. The net effect of encouraging the beneficial bacteria, such as Lactobacilli, and suppressing the pathogenic bacteria such as Salmonella, Shigella, Staphylococcus, Escherichia coli, Clostridium and Helicobacter pylori, is greater body weight and improved feed efficiency and healthier animals with fewer antibiotics.

### BRIEF DESCRIPTION OF THE SEVERAL VIEWS OF THE DRAWINGS

FIG. 1 is a front view of an installed water system in accordance with one embodiment of the present invention.

FIG. 2 is an exploded view of the holding chamber, the electrocatalytic cell, and the flow switch assembly in accordance with one embodiment of the present invention.

FIG. 3 is a cut away view of the electrocatalytic cell plate assembly contained within a housing shown in FIG. 2 according to principles of the present invention.

FIG. 4 is an enlarged view of one embodiment of an electrocatalytic plate configuration for use in the housing shown in FIG. 3 according to the principles of the present invention.

FIG. 5 is an enlarged isometric view of one embodiment of the electrocatalytic plate rail for use with the electrocatalytic plates shown in FIG. 4 according to the principles of the present invention.

FIG. 6 is a plan view of the plate rail shown in FIG. 5 according to the principles of the present invention.

FIG. 7 is an enlarged isometric view of one embodiment of the housing shown in FIG. 3 for the electrode plates according to the principles of the present invention.

FIG. 8 is a schematic view of the holding chamber gas release assembly coupled to the collection valve as shown in FIG. 2 according to the principles of the present invention.

FIG. 9 is a schematic view of another embodiment of an installed water system in accordance with one embodiment of the present invention.

FIG. 10A is a schematic line drawing of one embodiment of a prefiltration section of the water system in accordance with one embodiment of the present invention.

FIG. 10B is a schematic line drawing of one embodiment of a water softener and cell input section of the water system in accordance with one embodiment of the present invention.

FIG. 10C is a schematic line drawing of one embodiment of a cell output and pressure tank section of the water system in accordance with one embodiment of the present invention.

FIG. 11 is an exploded view of the power connections to the electrocatalytic cell and close switch shown in FIG. 1 according to the principles of the present invention.

FIG. 12 is a schematic view of one embodiment of the face panel of the controller shown in FIG. 1 according to the principles of the present invention.

### DETAILED DESCRIPTION OF THE INVENTION

A water treatment system, and in particular, an apparatus and a method for treating water for use with livestock and poultry, is described in detail below. In the following description, numerous specific details are set forth, such as example environments, contaminants, configurations and material selection, etc., to provide an understanding of the invention. One skilled in the relevant art will readily recognize that the invention can be practiced without one or more of the specific details, or may be practiced to treat water in a variety of situations and applications. Well known structures or operations are not shown or described in detail to avoid obscuring aspects of the invention.

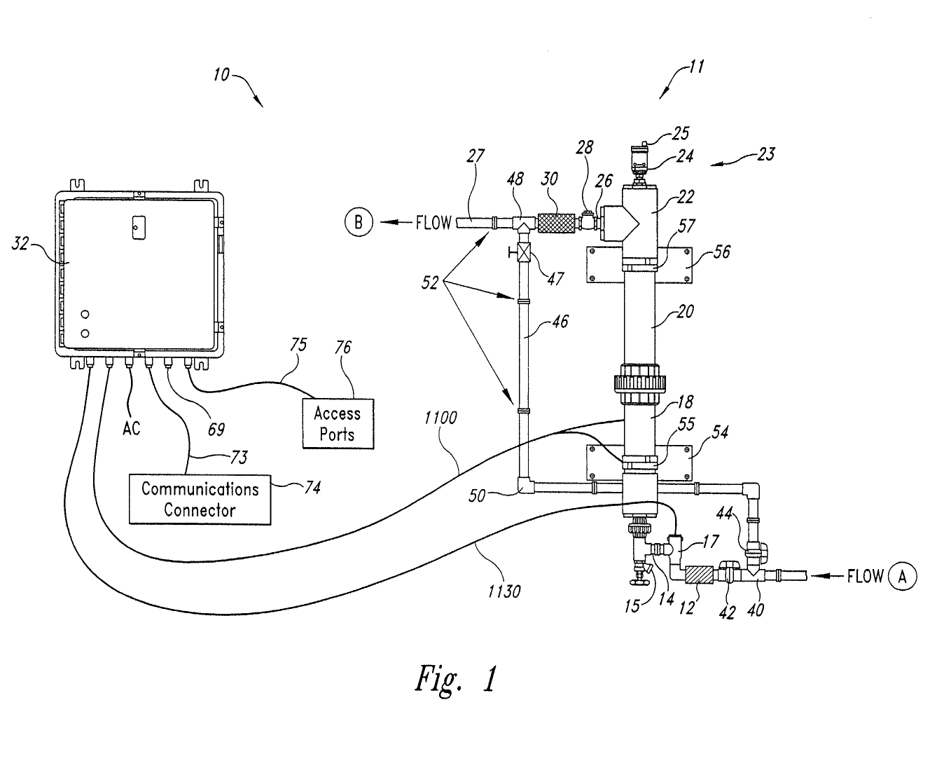

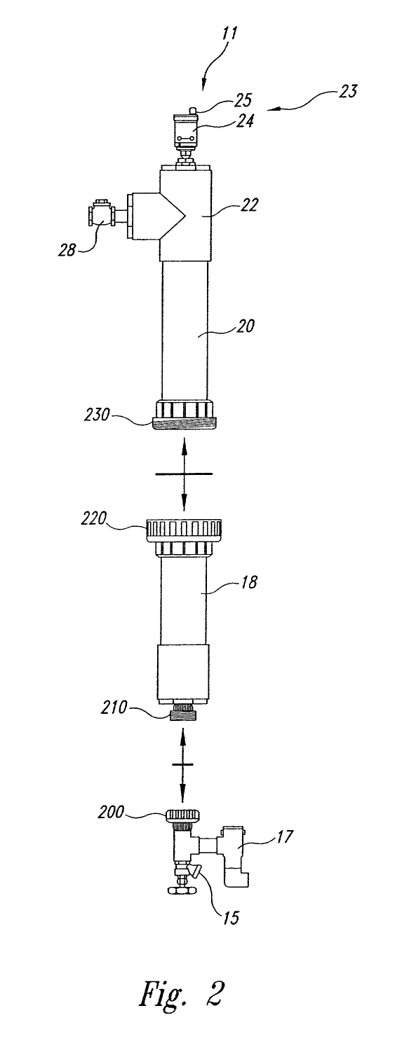

FIGS. 1 and 2 illustrate a water treatment system 10 wherein water is input in the system at “A” and water is output out the system at “B” for use in a livestock drinking system.

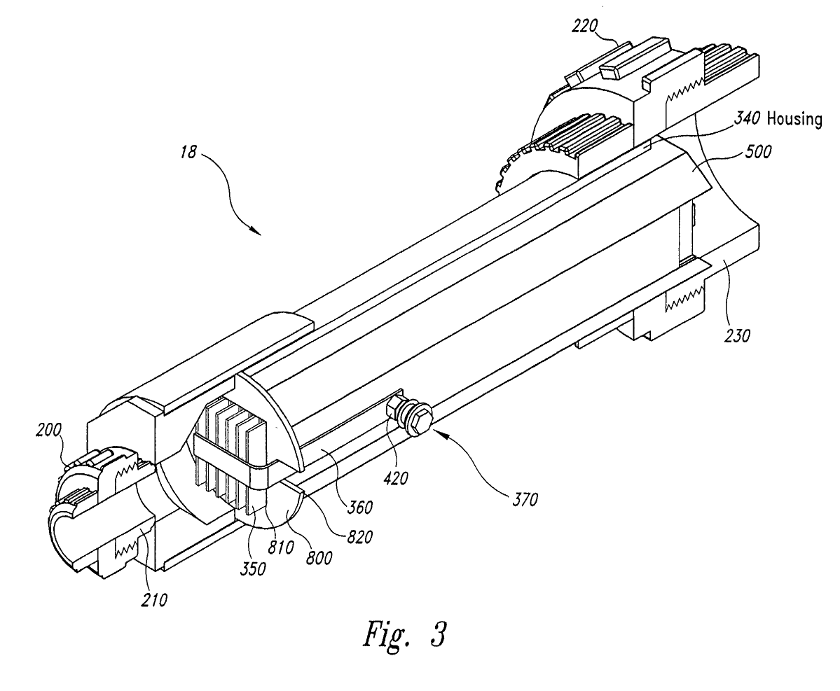

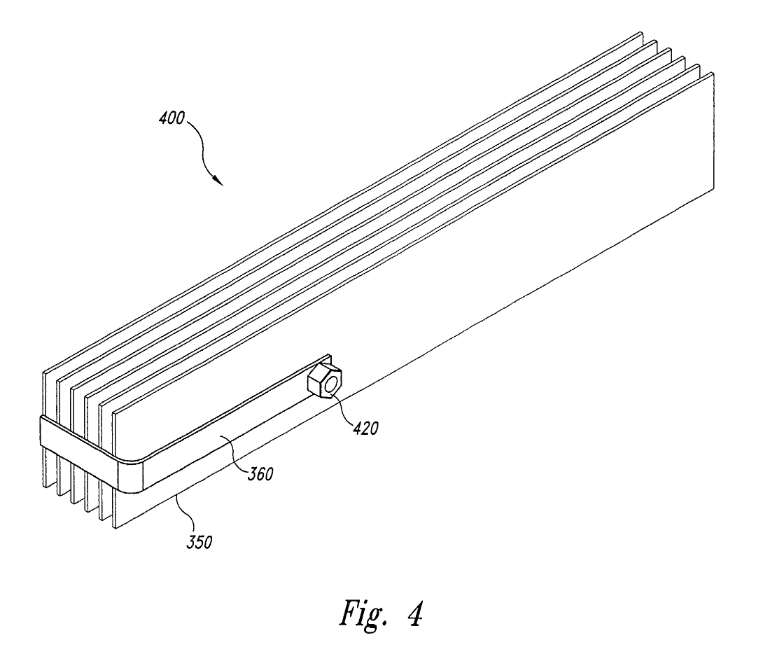

The components of the system will now be described in more detail with respect to FIG. 1. FIG. 1 illustrates one layout of the water treatment system 10. In this embodiment, water enters the system 10 at point A, coming into the system 10 under pressure, typically from a pressure tank, a pump, or line pressure from a municipality. The water progresses from entry point A through a treatment filter 12, through a flow meter 17, and into a treatment unit 11. The treatment unit 11 includes an electrocatalytic cell 18. The water flows across conductive plates (shown in FIGS. 3, 4) in the electrocatalytic cell 18 and into a holding chamber 20. An upper portion 22 of the holding chamber 20 includes a gas release assembly 22 that allows gases to egress through a gas vent 25 in a collection valve 24 positioned at the top of the gas release assembly 22. The gas release assembly 22 includes an outlet 26 to allow the water to flow out the outlet 26 of the upper portion 22 of the holding chamber 20 and through a check valve 28. The check valve 28 prevents backflow of water into the treatment unit 11.

After passing the check valve 28, the water proceeds out the exit piping 27 and through an optional sediment filter 30 exiting the water treatment system 10 at point B ready to be used in a livestock drinking system of any acceptable type. Typical livestock drinking systems for poultry include drinker lines with stainless steel nipples for the birds. Depending on the ultimate use of the treated water, pressure reducer valves may additionally be included in the system. The water treatment unit 11 should be positioned on the high pressure side of any pressure reducers.

Prior to entering the treatment unit 11, a tee 40 may be positioned in the pipe. Valves 42 and 44 may be opened and closed as desired to direct the flow of the water. To direct the flow of the water through the treatment unit 11, valve 42 is opened and valve 44 is closed. Alternatively, valve 42 can be closed and valve 44 opened to allow the water to flow around the treatment unit 11 and into the livestock drinking system as untreated water via piping 46. The water re-enters the treated water line at a pipe tee 48 and then flows through exit piping 26 to the livestock drinking system.

In one alternative embodiment, a valve is placed in line 46 though it is not present in a first design. The valve in line 46 prevents water from flowing back into the bypass piping 46. It can be a check valve or a controlled valve that is opened and closed as desired. Similar to the opening and closing of valves at the inlet of the system, when the bypass piping 46 is being used, check valve 28 is closed and its valve is open. Alternatively, when the treatment unit 11 is being used to process the water, check valve 28 is open to permit flow out of the system, but will prevent flow in reverse back into the treatment unit 11 and the valve in line 46 is closed.

This bypass piping configuration allows the user to choose between treating the water by closing valve 44 and opening valve 42 and thereby running the water through the treatment unit 11 or alternatively bypassing the treatment unit 11 by opening valve 44 and closing valve 42 to allow the water to bypass the treatment unit 11 entirely. This will be advantageous to allow routine maintenance or replacement of parts on the treatment unit 11. Standard elbows 50, tee's 40, 48 and union pipe couplings 52 appropriate for the gage, size and type of the piping used will provide connections for the water line.

The treatment unit 11 is mounted in a vertical position with mounting brackets 54, 56 and corresponding pipe clamps 55, 57 or similar securing hardware. Rigid pipe connections entering and exiting the treatment unit 11 encourage a secure and fixed mounting of the treatment unit 11.

The treatment filter 12 is placed, in one embodiment, after valve 42 and prior to flow meter 17. The filter 17 removes materials that make the water hard. One example of a treatment filter is a conventional water softener system such as a cell resin bed softener filled with salt. Duplex systems may be used wherein one tank is regenerating while the other tank is treating the incoming water. It will remove calcium, magnesium and other hardness minerals from the water to create soft water. The calcium and magnesium atoms are replaced with sodium to make the water soft, as is well known in the water softener art. The filter 12 may also be placed at inlet point A, prior to the bypass piping.

Yet another alternative treatment filter 12 that may be used is a reverse osmosis (R/O) system. The RIO system includes a semipermeable membrane which forms a very tight filter through which the incoming water must flow. Increased pressure pushes the water through the semipermeable member while wash water across the membrane keeps the membrane clear of contaminants. Bacteria is removed in this system down to the micron level. Further, under the R/O system, the water conductivity is taken down to 10 microsemens. An R/O system removes nearly all minerals and all other impurities from the water and thus results in very clean, pure water.

Yet another treatment filter 12 that may be used in the system 10 are magnets affixed to the water pipes in combination with a water softener or alone. Magnetically induced resonance (MIR) devices include a series of powerful magnets. These affect the mineral clusters and cause them to remain in solution by changing the electrical properties of the molecules of the mineral and/or the water. In combination, the water softener could remove some portion of the minerals and the magnets could alter the properties of the remaining materials such that they remain in solution and pass through the system 11.

The water filter 12 is not shown to scale in correct size, but is shown only schematically. As will be appreciated, a conventional water softener using a resin bed with salt may be much larger in size than the treatment unit 11 or electrical control box 32, particularly if a duplex tank system is used. The reverse osmosis system is also of a different size and includes further piping as is known for such filters.

The purpose of filter 12 is to standardize the mineral content and electrical properties of the water prior to treatment. As is known, some water is very hard and thus is high in calcium, magnesium, iron or other minerals. If these pass into electrocatalytic cell 18, they may cause build-up and scaling on the plates. They may also shorten the life of the plates and affect their electrical properties over time. It is therefore desired to remove from the water those compounds and elements that will cause clogging of the plates or decrease the effectiveness of the plates over time.

After the water treatment filter 12, water flows through a flow meter 17 whose electronic signal output is connected to a control unit 32. The data from the flow meter 17 is used by control unit 32 to control the current provided to the cell 18 based on the flow volume registered by the flow meter 17. Current density is regulated through the electrocatalytic cell 18 and is increased or decreased proportional to the flow as measured by the flow meter, as explained in more detail later herein.

The flow meter 17 can be any acceptable type. It can be based on an ultrasonic sensor, a doppler shift technique, a Hall effect sensor, a mass flow meter, or any acceptable type of flow meter used for water or other liquids. The flow meter 17 outputs an electronic signal indicating the flow rate of water through it. The output can be analog or digital. The signal is received by the controller 32 and used for current density control of the plates, as explained herein.

The water flows through the flow meter 17 and into the inlet 14 of the treatment unit 11. A magnet or MIR device can be placed around the inlet 14 if desired, just prior to the cell 18. Below the inlet 14 in the treatment unit 11 line is a discharge valve 15. The discharge valve 15 may be opened to drain the treatment unit 11 in cases of routine maintenance or alternatively to drain sediment out that has precipitated out and settled to the base of the treatment unit 11.

Water flows up through the electrocatalytic cell 18 and proceeds into the holding chamber 20. The holding chamber 20 is a separate unit coupled to the electrocatalytic cell 18 via a standard connection. As will be appreciated, an optimum length 1 of the holding chamber is related to the flow rate desired, as well as the diameter of the holding chamber 20 and other factors. Alternatively, the diameter of the holding chamber 20 may be made larger than the diameter of the cell, providing a slower flow rate and longer resident time for the same volume of flow rate. One preferable ratio of volume of water between the holding chamber and the cell chamber is 7:1.

Water proceeds from the holding chamber 20 into an upper portion 22 of the holding chamber 20 that included a gas release assembly 23. In the upper portion 22 of the holding chamber 20, the water in the treatment unit 11 proceeds out the outlet 26 while any gases are collected in the gas release assembly 23. Opening a collection valve 24 allows gases to vent out a gas vent 25. At the outlet 26 of the treatment unit 11, a check valve 28 prevents water from back-flushing the system. Water proceeds out the exit water piping 27 into the sediment filter 30.

The sediment filter 30 prevents sediment from entering the livestock drinking system. The sediment filter 30 can be a self-cleaning, back-flush type, filtration device. Filters of this type operate via a backwash cycle that cleans the filter without interrupting the main system water flow. These filters operate on line pressure alone. Alternatively, the sediment filter 30 may be a simple course screen that requires periodic cleaning or replacement. Typical livestock drinking systems for poultry include drinker lines with stainless steel nipples for the birds. These stainless steel nipples are easily clogged by sediment, and if clogged, require intensive manual labor to individually replace or unclog. Clogged feeder lines left unattended can cause flooding of the drinker lines and wet litter in the feeding area that can cause disease to the birds or leg problems.

The electronic controller 32 includes a microprocessor, a memory, a power supply circuit and all other electronics needed to control and monitor the electrolytic cell. Power is provided into the controller 32 as AC power in. The controller 32 includes an AC to DC converter to generate a DC output of the proper voltage and current values. A signal line 1130 carries the output from the flow meter 17 and inputs it to the electronic control circuit 32. The data from the flow meter is used to determine the voltage and current to be provided to the electrolytic cell via power supply line 1100. A communications connector 74 for connection to the outside world is provided via line 73. Other access ports 76 can be connected via line 75. An additional port 69 is provided for future peripheral expansion as desired. Further, port 75 may also be used for further peripheral expansion as needed.

FIG. 2 illustrates an exploded view of the treatment system 11. The system is easily disassembled into three main components. The first component includes the flow meter 17, the discharge valve 15 and a union joint 200. The union 200 mates with a union 210 in a second component. The second component includes the union 210, housing for and the electrocatalytic cell 18 and a second union 220. The second union is positioned at the top of the electrocatalytic cell 18 and mates with a second union 230 at the bottom of a third component. The third component includes a second union 230, the holding chamber 20, the upper holding chamber 22, the collection valve 24 with a gas vent 25 and the check valve 28.

The couplings 200 and 220 are female couplings and the couplings 210 and 230 are male. This is selected based on the direction of water flow. It flows from the male to the female to prevent water leakage and provide a tighter fit. The coupling connections used are a significant advantage in providing service and cleaning of the electrocatalytic cell assembly 18 and the housing 20. When it is desired to service the electrocatalytic cell 18, such as cleaning the electrodes 350, replacing or servicing any of the components or the like, operation of the system is terminated for a brief period of time. Water is drained from the apparatus. The couplings are then rotated so as to separate the electrocatalytic cell assembly 18 from the rest of the system. The cell housing 340 is thereafter removed from the system for replacement, servicing or the like if desired. Thereafter, the cell housing, having the new electrocatalytic cell or the cleaned cell therein is replaced and the couplings are reattached so the system becomes fully operational. The unions can be any acceptable coupling, including rotatable threads, watertight couplings or the like, many such watertight connections being known.

As constructed, the treatment system 11 is easily assembled and disassembled in the field. For example, the various components, including the flow switch and discharge valve, the electrocatalytic cell, and the holding chamber are connected with easily releasable fittings for fasteners such that a user can disassemble it. It also includes easy-to-assemble connectors such that a user can quickly assemble it in the field or perform a reassembly after the cleaning. For example, the coupling between the electrocatalytic cell 18 and the holding chamber 22 is preferably an easy-release and easy-assemble-type coupling. An example of this type of union includes a threaded union, snap-on clamp, rubber gasket seals or other couplings that can easily be assembled and disassembled. In the embodiment shown in FIGS. 1 and 2, the coupling is a threaded coupling using standard threaded fittings between the cell housing and the holding chamber. Other acceptable, and equivalent, coupling techniques can be used so as to provide easy disassembly in the field for cleaning and maintenance, and also permitting easy assembly and reassembly so the unit may be put back into service by a general worker that does not require special skills or training in this particular technical field. Further, it can be reassembled and put back into service in a very short period of time following such disassembly for cleaning.

In one embodiment, a pressurized system is used to increase the level and consistency of dissolved oxygen (DO) in the animal husbandry system described herein. The system may include a pressure tank employing a valve and venting device that maintains constant water level in the tank while allowing for continual venting of excess hydrogen and oxygen gases. The pressure system is plumbed in series between the electrolytic cell and dissolving chamber assembly and the water line feeding the animal watering system or drinker lines.

In operation, the pressure system is charged with water containing DO and entrained gas bubbles of gaseous oxygen and hydrogen created by the electrolytic cell. As the pressure tank fills, air is vented though the gas relief valve until the water reaches a float valve that closes the air vent passage. The tank continues to fill until the gas pressure above the water equals the incoming water pressure. Once filled, the water level remains relatively constant over a wide range of flow rates. Hydrogen and oxygen gas released from the water collect in the headspace above the water in the tank. Due to the large difference in density between oxygen and hydrogen, the gases tend to stratify providing an oxygen rich layer in contact with the water surface. Since the solubility of a gas is directly proportional to the partial pressure of the gas at the liquid to gas interface, the water becomes super saturated with DO. A second source of increased DO results from the increased residence time that the tank volume provides. The latter residence time allows sufficient time for the transfer of entrained oxygen gas bubbles into the dissolved state prior to being released to the animal watering system. Excess hydrogen gas is continually released from the top of the tank cavity to maintain a relatively constant water volume in the pressure tank

The advantages of this embodiment include increases in DO levels of 3 ppm above those levels currently produced by an unpressurized water treatment system of the present invention. Further, use of the pressurized system provides consistent DO levels and helps to compensate for variables such as varying flow rates and cell electrode performance deterioration.

FIG. 3 illustrates an enlarged view of one embodiment of the electrocatalytic cell housing assembly 18 with the housing of the cell partially cut away to reveal the cell assembly configuration within. In one embodiment, the housing 340 and the couplings 310, 320 of the electrocatalytic cell are constructed from schedule 40 PVC. A diameter of ½″ can be sufficient, but for certain applications, the diameter may be 1′, 2′, 3′ or larger. As shown in the exemplary embodiment, couplings 200 and 210 are easy to assemble and can be either threaded or a pressure fit to connect the assembly 18 to the inlet pipe. The cell plate housing 340 may alternatively be constructed of a metal, such as the same material as the electrocatalytic cell plate electrodes 350. The electrodes 350 of the electrocatalytic cell are appropriately connected to the positive and negative power supplies via an L-bracket 360 and a connecting bolt 370, as is known in the art. The length and the number of the electrodes are selected so as to provide the desired amount of oxygen generation, again according to known principles.

The electrode plate assembly further includes a water block 800 at a top end of the electrode plates 350. The water block 800 includes an opening 810 for the electrode plates to pass through as well as a cut out 820 for the L-shaped bracket to seat in.

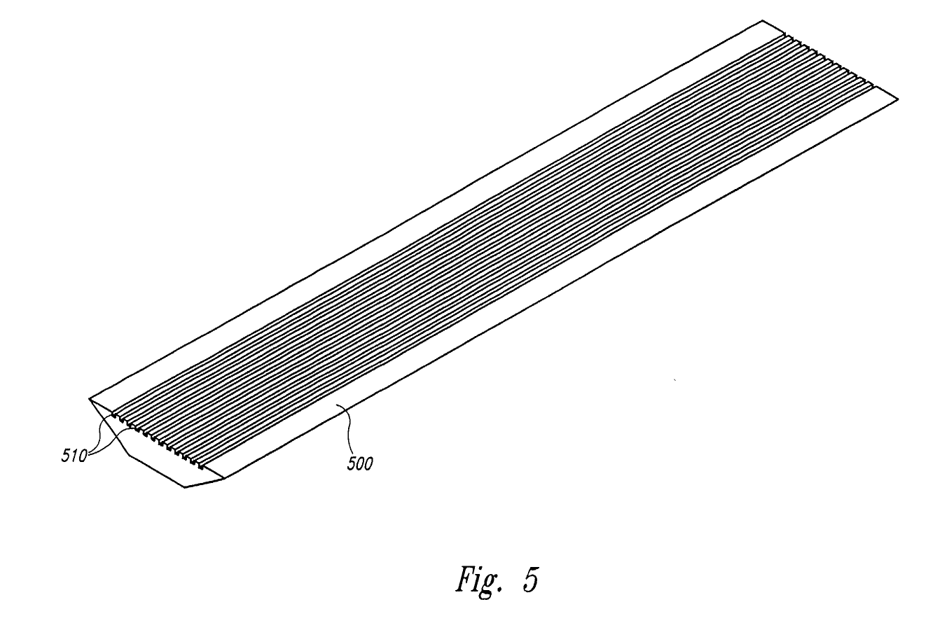

FIG. 4 illustrates the electrocatalytic cell plate assembly 400 housed within the housing 340 of FIG. 3. In the embodiment of the electrocatalytic plate assembly shown in FIG. 4, electrode plates 350 include anodes and cathodes. The anodes and cathodes can be coated in a double-sided EC-400, nickel, platinum, double-sided tin or stainless steel. In one exemplary embodiment, spacing between the charging plates is approximately 0.08 inches. Plate dimensions are 1 inch by 6 inches in one embodiment and 2 inches by 12 inches in an alternative design. They can also have other dimensions. According to one design, rectangular plates having the long face aligned with the flow direction are preferred. Plate configuration can be 4 to 12 electrode plates, such as a 12-electrode plate configuration shown in FIGS. 4 and 5.

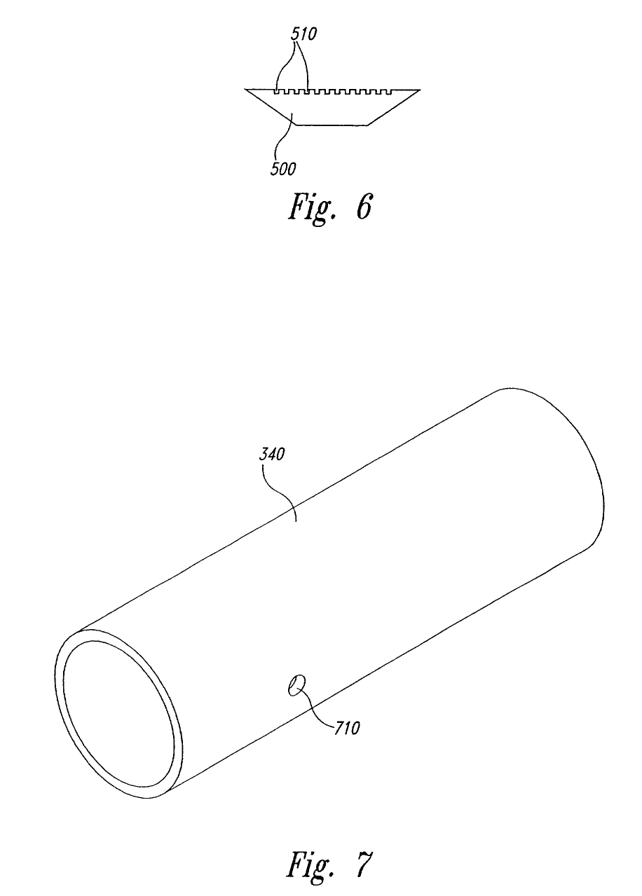

FIG. 5 illustrates a schematic view of the electrocatalytic cell plate rails 500 of FIG. 3. FIG. 6 illustrates a plan view of the electrocatalytic cell plate rails 500 of FIG. 3. The grooved plate rail 500 sandwiches an upper and lower side of the plates 350 wherein an edge of one charging plate extends into the grooves 510 of the plate rail 500. As shown in FIGS. 5 and 6, grooves 510 in electrocatalytic cell guide rails 500 hold the electrode plates 410 a preset distance apart from each other at all times. A titanium bolt 370 connects to L-shaped bracket 360 at each end of the plates 350. The bracket 360 in the illustrated embodiment is shown welded to the plates 350. Alternatively, the electrocatalytic cell plates may be bolted together or the bracket 360 may be at the top, horizontal to the flow direction. The bolt provides an electric connection to the charging plates 350 to conduct power to the cell for electrolysis to occur and extends through to the outside of the housing of the cell.

FIG. 7 illustrates a schematic view of one embodiment of the housing 340 for the electrocatalytic cell, showing a hole 710 for the titanium bolt to extend therethrough.

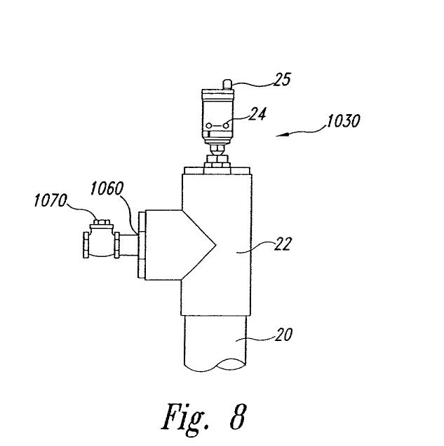

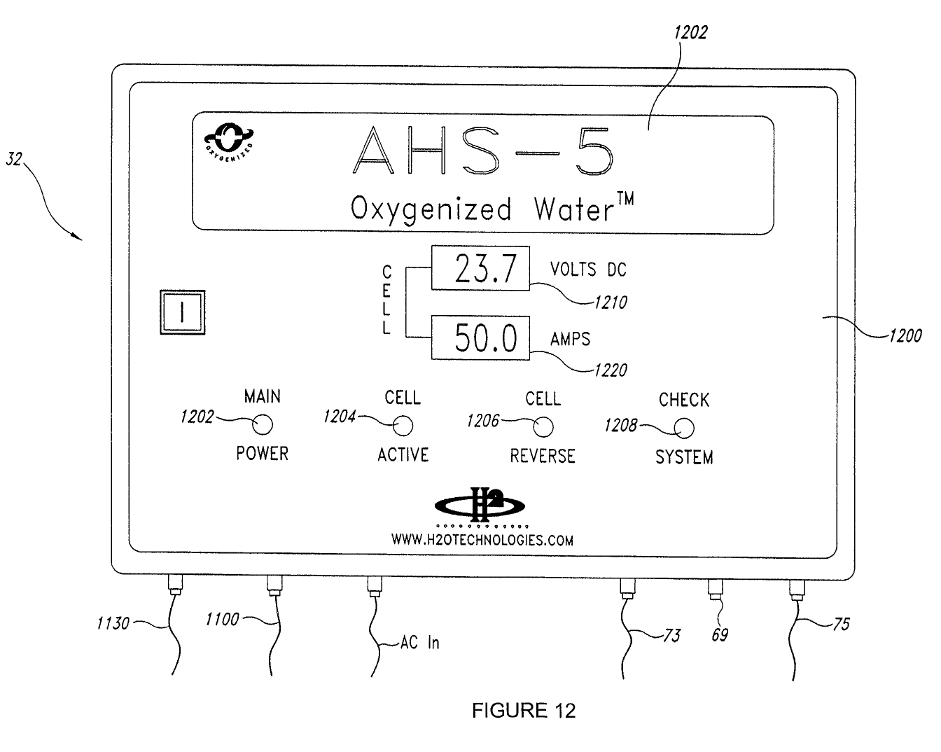

FIG. 8 illustrates a schematic view of the upper portion of the holding chamber in accordance with one embodiment of the present invention. According to one embodiment of the present invention shown in FIG. 12, the holding chamber 20 is a straight, longitudinally extending tube with an unrestricted cross-sectional area. The housing for the holding chamber 20 may be a clear glass tube, or may be constructed of the same material as the housing for the entire treatment unit. In one embodiment, the housing for the electrocatalytic cell 18 has the same cross-sectional diameter as the holding chamber 20 so as to provide a generally smooth, laminar transition from the electrocatalytic cell to the holding chamber. Generally, the holding chamber 20 will begin immediately above the electrocatalytic cell so that the generated oxygen gas can begin to transition into the dissolved state. In an alternative embodiment, the holding chamber 20 has a larger diameter than the cell housing to provide an extended resident time for a given flow rate. One end of the holding chamber includes a coupling having threads for connecting to the electrocatalytic cell housing.

An upper end 22 of the holding chamber 20 includes a gas release assembly including a collection valve 24 with a gas vent 25. The gas release assembly allows release of accumulated gas. The upper portion 22 of the holding chamber 20 also includes and outlet 1060 for the water and a check valve 28 in line with the outlet 1060. The check valve 28 prevents backflow of untreated water into the treatment system when the bypass piping is being used. The check valve 1070 also prevents treated water from backflushing into the treatment system when cleaning filter 30 or at other times.

The unrestricted cross-sectional area of the holding chamber of the embodiment shown in FIG. 8 permits water to pass therethrough in laminar flow without encountering obstructions. This provides a quiet zone, which permits the oxygen molecules to more easily be dissolved into the water. If the holding chamber is made too short, the housing will terminate before a majority of the oxygen has dissolved into the water and will thus be exposed to surface air and exit in the gaseous form, rather than becoming dissolved in the water. Further, if turbulence is induced in the water, such as by having a sharp turn, a 90° elbow, or other obstructions immediately after the cell before sufficient quiet time has been permitted, then the oxygen and hydrogen will be inclined to remain in the gaseous state and not transition to dissolved oxygen.

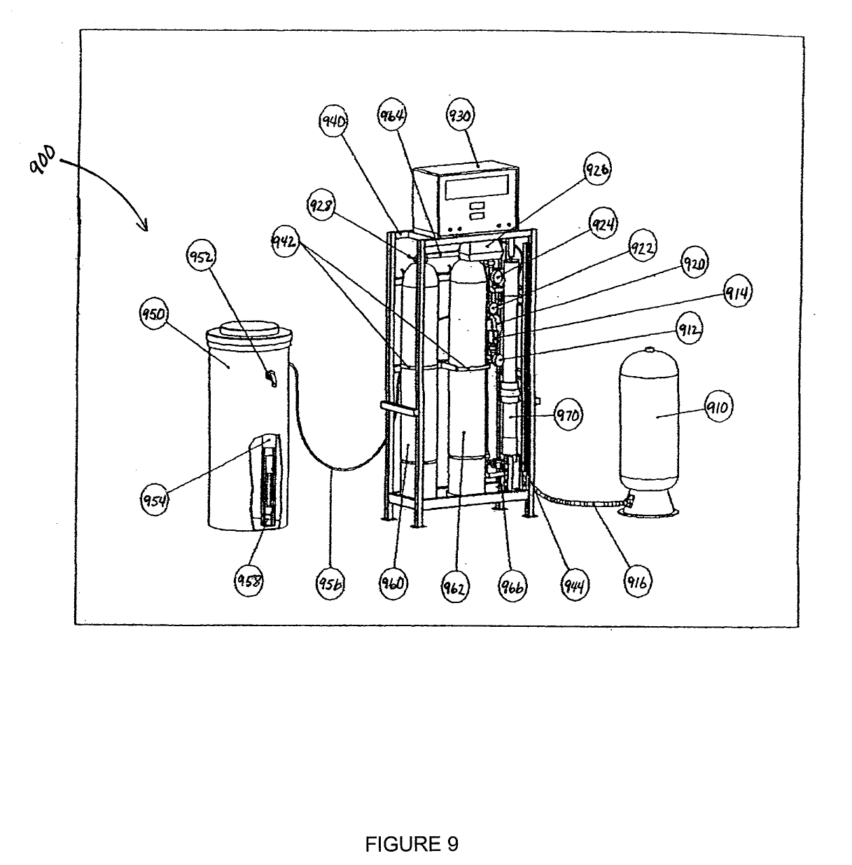

FIG. 9 illustrates a schematic view of another embodiment of an installed water system. The system 900 is shown mounted on a skid 940 which may be made of stainless steel or other suitable materials and secured in place with straps 942 for transportation. A controller 930 controls the system 900 and records data as described in greater detail herein. The system 900 includes a pressure system comprising a pressure tank 910, pressure switch/gages 912, 914, and a pressure vessel interconnect line 916. A water hammer arrestor 920 is positioned downstream of the pressure system and is monitored by to pressure switch/gages 922, 924. Two softener resin tanks 960, 962 are shown in the present embodiment. The tanks 960, 962 are connected and regulated via a water softener interconnect line 964, a valve head assembly 926, 928 and a flow meter 966. An electrolytic cell 970 is positioned downstream of the water softener tanks. The system 900 may further optionally include a brine tank 950. In the present embodiment, the brine tank 950 has an overflow 952, a brine well 954, a flexible brine suction pipe 956 and a brine valve 958.

The illustrated embodiment includes a 20-liter/minute single pass electrolytic cell 970 followed by a mixing chamber. A flow meter measures water demand and generates a signal to the controller that provides the appropriate power to the electrolytic cell. The controller manages the operation of the system, stores operating data and allows the periodic downloading of information for operations or maintenance analysis. In some embodiments, a water softener is required ahead of the cell to prevent the build-up of scale that could otherwise impair cell performance. The pressure tank of the illustrated embodiment is 112 liters. The brine tank of the illustrated embodiment is 250 liters.

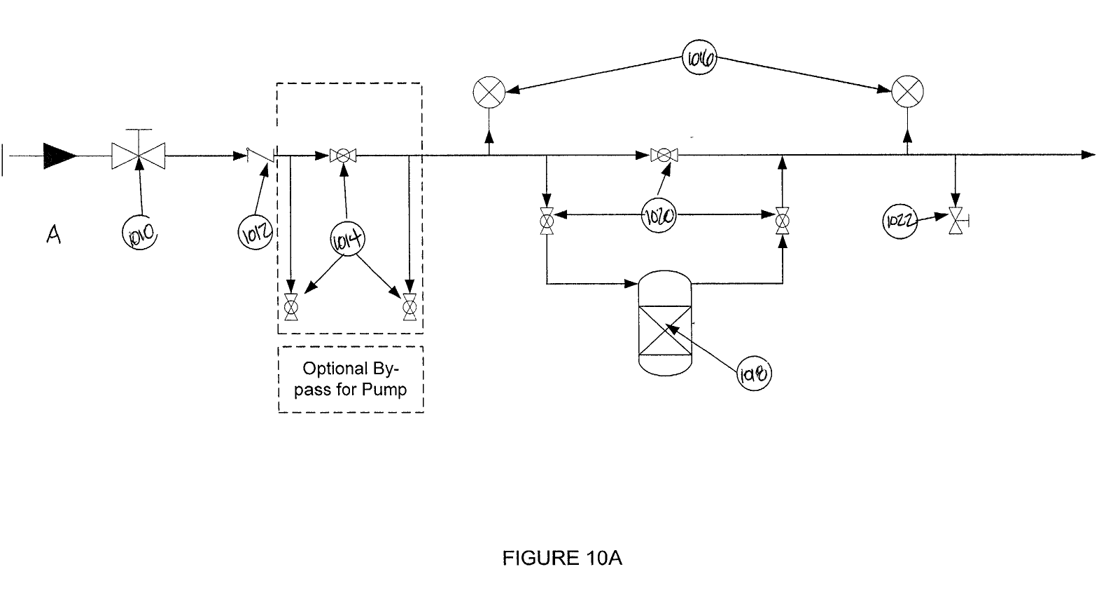

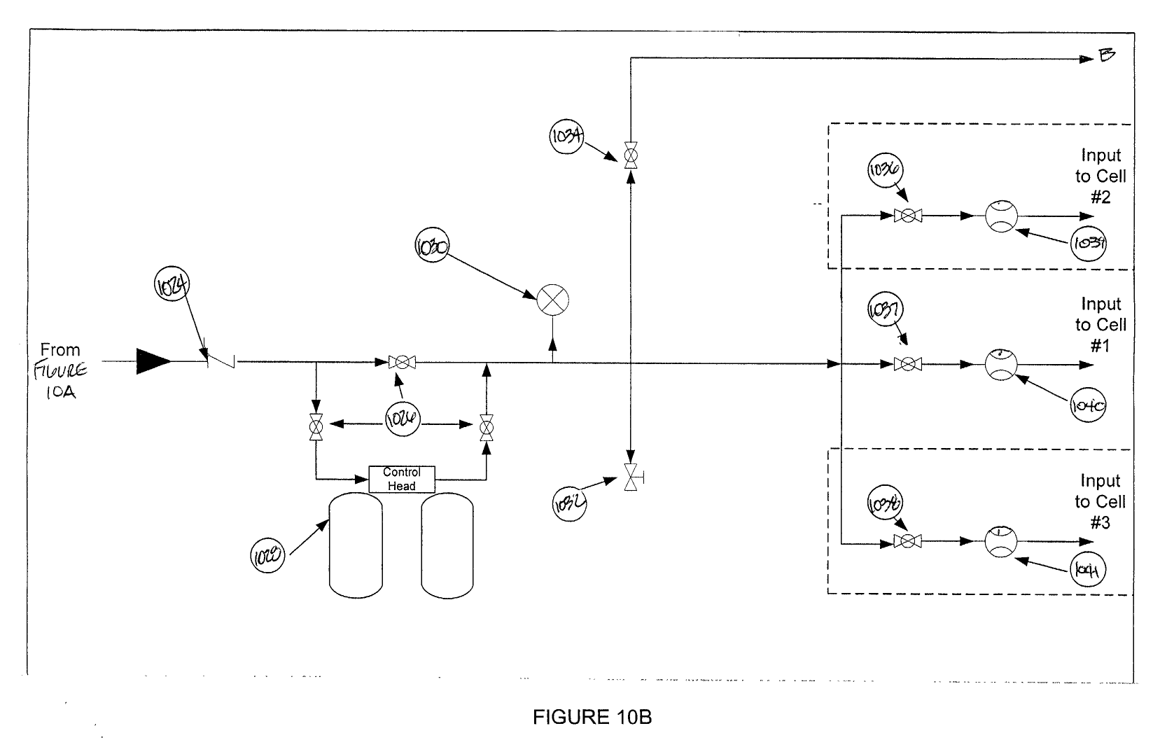

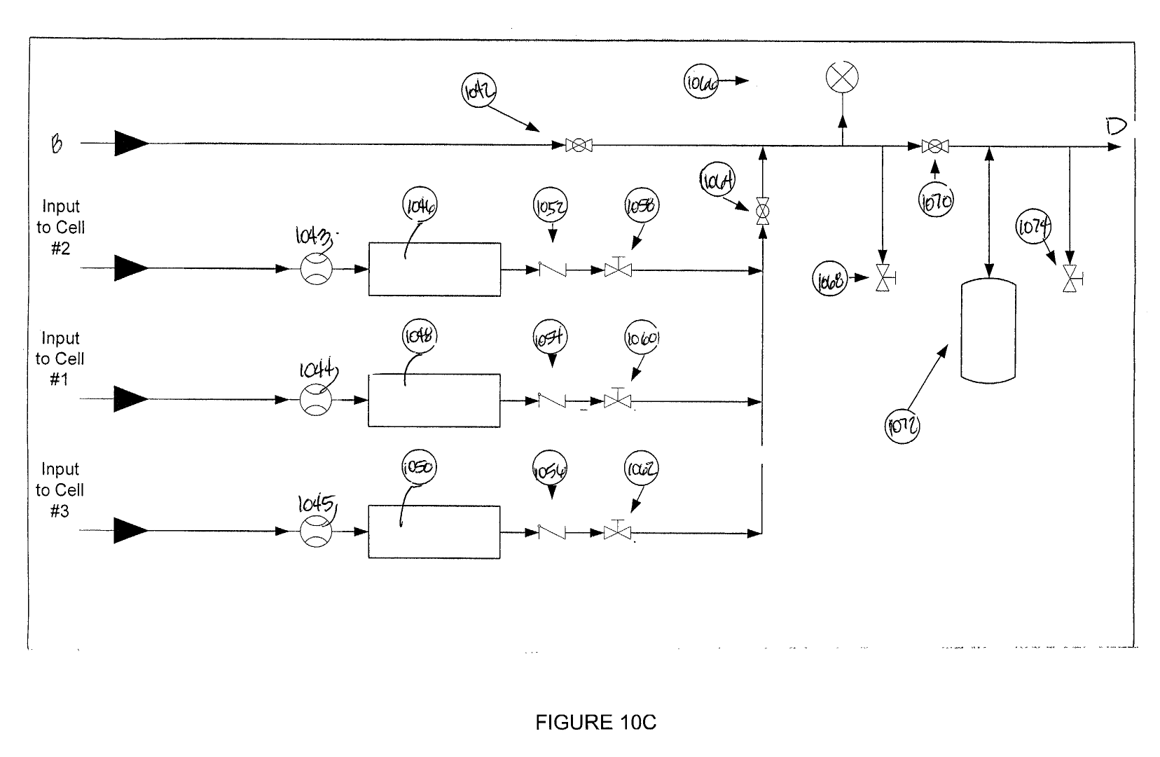

FIGS. 10A, 10B and 10C illustrate a schematic line drawing of one embodiment of a prefiltration, softener and cell input, and cell output sections of the water system. FIG. 10A illustrates incoming water A flowing through a pressure reduction valve 1010 set to a maximum of 60 psi. The water then flows through a spring valve 1012 and through a series of ball valves 1014. The ball valve configuration allows an optional by-pass for a pump to be installed in the system (shown in dashed lines). A pressure gauge 1016 is positioned in line above and below a filter 1018. A second configuration of ball valves 1020 allows the water to by-pass the filter 1018. As the water exits to the softener, a T is placed inline and capped with a valve and tap 1022 to provide a fresh water sample location.

FIG. 10B illustrates water incoming from the prefiltration section of FIG. 10A. The water flows through a spring valve 1024 and through a configuration of ball valves 1026 that allow the water to flow through a twin bed softener or to bypass the softener 1028. A pressure gauge 1030 is positioned downstream of the softener 1028. A connection the allows the water to flow to a valve with a tap 1032 for taking a soft water sample, through a ball valve 1034 and out to a water bypass, or into the electrolytic cells. This embodiment illustrates a system with three electrolytic cells. Valves 1036, 1037, 1038, control which cell the water will flow through. Valve 1037 may optionally be a main cell shut off valve. Downstream of the valves are flow meters 1039, 1040, 1041.

FIG. 10C illustrates water incoming from the softener and cell input section of FIG. 10B. The bypass water B flows through ball valve 1042 and does not flow through the electrolytic cells. Depending on the open/close configuration of the ball valves 1036, 1037, 1038, water will flow through flow meters 1043, 1044, 1045 and into electrolytic cells 1046, 1048, 1050. Upon leaving the electrolytic cells 1046, 1048, 1050, the water will flow through spring check valves 1052, 1054, 1056 and then through balancing valves 1058, 1060, 1062 and into a combined line and through ball valve 1064 to join any bypass water. A pressure gauge 1066 registers the pressure of the treated water and the bypass water. Downstream a T is placed inline and capped with a valve and tap 1068 to allow a treated water sample to be taken. A shut off valve 1070 may optionally placed in line prior to a pressure tank 1072. A downstream T allows yet another valve and tap 1074 for taking a post pressure water sample prior to the water flowing into the drinker lines D.

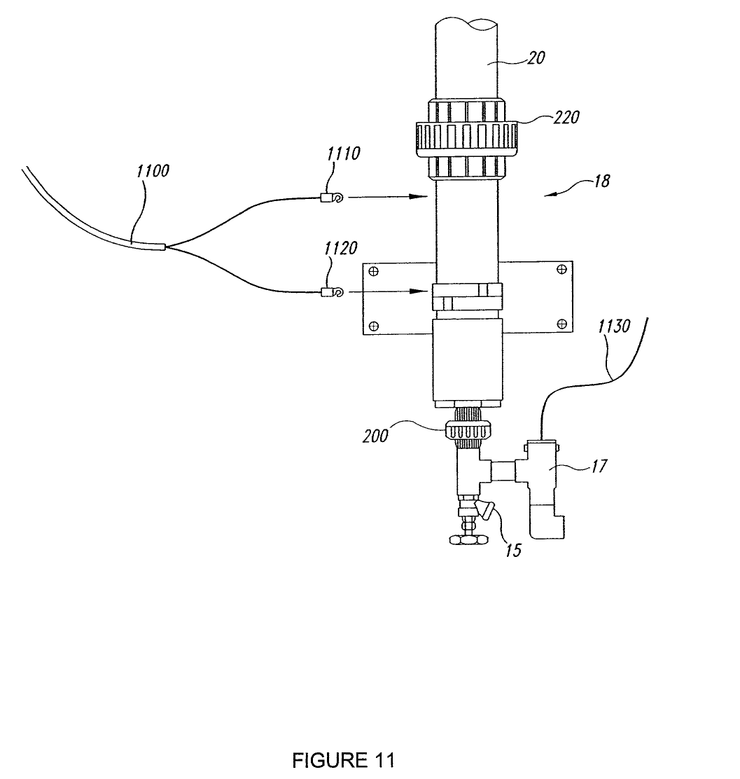

FIG. 11 illustrates a schematic view of the electric connections to the electrocatalytic cell and flow switch in accordance with one embodiment of the present invention. Electric connections 1110, 1120 are provided from the control panel 32 (shown in FIG. 1) to the bolts on the side of the electrocatalytic cell. Further, a signal connection 1130 extends from the control panel to the flow meter 1. Current density applied to the electrocatalytic cell is adjusted by the electronic controller based on the flow volume measured by the flow meters as explained herein.

FIG. 12 illustrates a schematic view of the control panel 1200 of the electronic controller 32 of FIG. 1 in accordance with one embodiment of the present invention. The control panel includes a main power indicator 1202, a cell power indicator 1204, a reverse polarity indicator 1206, and a check system indicator 1208. These indicator lights allow the user to quickly verify the status of the system in operation and to identify any potential problems. In addition to identification indicators 1202, 1204, 1206, 1208 the control panel 1200 includes a voltage output 1210 and amperage output 1220 reading for the power provided to the electrocatalytic cell.

Power to the electrocatalytic cell in the present embodiment includes standard cabling that is approximately 10-15 feet in length with ring terminals soldered on. It can be enclosed in a water resistant coating and be connected with a rubber protective boot if desired. The cell power wire in the present embodiment is 6 gage or equivalent to provide sufficient capacity. For example, two 10 gage wires may be used, etc. The enclosure shown meets NEMA 4X rated standards. The control panel further includes a fan to extract internal heat and prevent heat buildup.

In one embodiment of the present invention, the modular power controller receives input power of 100 VAC/220VAC, 47-63 Hz Universal input. The AC line current draw is preferred to not exceed 20 Amps. This exemplary embodiment is designed to control a single electrocatalytic cell. Alternatively, multiple cells can be run from one modular power control unit that has a larger amp output. Operating at 50 amps, up to five gallons per minute (gpm) can be processed. Current provided to the cell is regulated by the flow meter 17, with a digital signal output. The sensitivity range of the unit is 50 amps divided by 17 segments. Amperage will therefore be adjusted proportionally with flow every 3 amps or 0.3 gallons per minute (gpm).

According to principles of the present invention, the signal output of the flow meter 17 is used by the electronic controller 32 to supply the proper current to the electrolytic cell 18 to maintain the dissolved oxygen output at a selected level. Assume, for example, that the flow rate is two gallons per minute. The electronic controller has stored a previously created table and software program so that given a flow rate over the electrolytic plates of the size and shape within the cell 18, a selected amount of current is provided to generate additional dissolved oxygen. This is a table which is previously stored, and empirically chosen according to known principles as can easily be done by those skilled in this art of generating dissolved oxygen from electrolytic plates having a given size, current density and spacing between them based on the water flow. According to one embodiment, 10 amps are provided for each gallon per minute, so a 2-gpm flow would cause 20 amps to be provided to the plates of cell 18. If the water flow increases, for example to three gallons per minute, then the current density across the plates will also increase as needed to continue to create treat water passing through the plates. As the water flow rate continues to increase for example to five gallons per minute or any values therebetween, the current density provided on the plates will correspondingly increase, or decrease as needed as correlated to the flow rate to produce the desired water treatment. In some embodiments, such a table may be linear, however, alternatively, depending on the composition of the plates and their aging, it may not be linear for given flow rates over long periods of time. Alternatively, rather than having a table stored which provides a set output based on a given flow rate, a formula may be used which provides a continuous change in the electronic output based on variations in the flow rate on a continuous basis. Thus, even very small changes in the flow rate will be input to the calculation and the appropriate changes made in the power provided to the electrolytic cell. All such feedback control systems fall within the concept of the present invention in which the output of a flow meter is monitored and the value used to determine the current density to provide to the electrolytic cell.

The control unit includes a soft start circuit and a soft power change circuit. The soft start circuit allows current to ramp up from its initial “off” condition to the specified value period of several seconds to provide even current dispersion across the electrode plates. The soft start circuit operates as follows. When the control unit activates the cell to begin passing current between the plates and through the water, an initial turn-on signal is generated. Indicator light 1204 indicating that cell is active is illuminated. A ramp is established starting at zero and having a desired slope. The power will increase gradually and will be stable at the desired amperage level after a certain period of time. The rate at which it will slowly approach the final current value can be selected as desired, preferably over the range of three to ten seconds and, in one embodiment is about five seconds. The current provided to the cell will slowly increase from zero towards this final value at a consistent rate. This use of the slow start is extremely helpful in increasing the life of the electrolytic cell. The current will have time to be evenly distributed across the plates and through the water. Rather than providing power with a sudden on switch, using a step change in voltage as was done in the prior art, the use of the ramp will cause the current density to slowly increase across all the plates and give sufficient time for the current density to equalize between all plates and create a uniform current flow through the water to begin electrolysis. This preserves the life of the plates and avoids sudden hotspots as may occur if a step change in voltage is placed on the plates when initially switched on.

The soft power change circuit operates when power is on and is changed from one value to another value. If current is being provided, for example, at one amp and is going to be increased to two amps, the change will be in the form of a ramp that slowly moves from one amp to two amps. This ramp slope is preferred to be more gentle than the soft start ramp and will change the power more slowly. For example, the ramp will be as such that it may take 20 to 30 seconds to change from one amp to two amps.

The control 32 thus has two soft current change circuits. The first is a steeper slope that is used to place current on the plates from a no-power mode to power on mode and the second is a different, less steep slope that changes the power from an existing on current flow to a different current value. Of course, such soft start and change circuits are optional and need not be used. If present, they improve performance of the device and extend the life of the cell.

The control unit further includes a circuit for polarity reversal in the cell output. This allows the user to reverse polarity and clean or de-scale the cell as needed. The interval at which polarity reverses to the cell will be user selectable within a range. Four jumper positions are provided on the control unit CPU card. The ranges can be set in software and changed by replacing the main IC chip on the control card. Therefore, an infinite timing ability with respect to polarity is provided. The control unit will remove power to the cell for one minute prior to the polarity reversal.

The control unit will further include two serial ports 69 and 76 for future peripheral expansion. This will allow the user to later add in additional features such as a dissolved oxygen meter, a PH meter, a conductivity meter, an oxygen reduction potential meter, etc.

For example, according to one alternative embodiment, a dissolved oxygen meter is provided in upper portion 22, or other suitable location after the electrolytic cell. The dissolved oxygen meter senses the actual value of the dissolved oxygen and provides an electronic signal that is output to the controller 32. The controller 32 stores this value of the dissolved oxygen as empirical data. For those embodiments in which a dissolved oxygen meter is provided, this signal may be used as a feedback signal to the power supply to the electrolytic cell. In the event the dissolved oxygen is higher than the desired value, the power can be reduced so as to save power in achieving the desired value. Alternatively, if the dissolved oxygen is below the desired value, the power can be increased so as to increase the dissolved oxygen to the desired level. Since the cost, and difficulty of installing dissolved oxygen meters is quite high, they will not be used in all embodiments, nevertheless it may be desirable in certain installations to provide a dissolved oxygen meter and provide the feedback monitoring as has been described herein. Other meters, such as a PH meter, a conductivity meter, or other types of sensors providing electronic output may also be provided and have their outputs provided to the controller 32. The data may therefore be collected and used to modify the power provided to the electrolytic cell or other perimeters in the performance of the system. All such collected data will, of course, be time correlated and stored in the manner described with respect to the current, voltage and flow rate as detailed elsewhere herein.

The control unit has one external input (normally closed switch) for failure monitoring equipment. The control unit has the ability to monitor two cell failure modes. The first failure mode will be unable to reach 30% of the requested current output. The second failure mode will be overcurrent or current at the cell in excess of 55 amps. The failure of the first type can be characterized as failure for the current actually provided to the cell not reaching the value as directed by the controller 32. For example, for given flow rate as sensed from flow meter 17, the electronic controller will output a desired current density to be achieved at the electrolytic cell 18. The voltage is then increased, or decreased to the value needed to achieve this current density. As the voltage changes, the actual current provided to the electrolytic cells is sensed so as to get an accurate measure of the current flow for a given voltage. If the voltage reaches a maximum value, but the current is still so low as to not be within the range called for by the flow rate as sensed from the flow meter 17, then a first failure mode is indicated and stored in the memory. It will thereafter be downloaded via the communications connector 74 as described elsewhere herein. Alternatively, in the event the current becomes excessive for a given voltage, this will also be seen as a failure mode that is stored and monitored. For example, if the flow rate calls for a selected current and the voltage begins to increase to achieve such a current but which results in the current reaching its maximum value, then this would indicate a failure mode because the current has exceeded an acceptable maximum value. In one embodiment, this acceptable maximum value is 55 amps. As will be appreciated, this value can be sent at any other level as desired for each given application. Such a high current rate may indicate such factors as debris across the plates shorting them together creating a low resistance, high current path, and some other malfunction in the system or other short circuit. Similarly, an inability to reach the desired current for a voltage range may indicate that the resistance of the water is at some unacceptably high level, that scaling has built up on the plates so as to increase the resistance to the current flow from one plate to another or some other factors. By monitoring the two types of failure modes, the electronic controller is able to confirm that the system is operational within acceptable perimeters at all times and, in the event it becomes non-operational can transfer a signal immediately via the communications connector 74 as well as illuminate check system light 1208.

In operation, the communication package in the controller allows recordation and retrieval of data via, for example, an EEPROM. Amperage, voltage, flow rate and failures can be recorded and data may be saved up to thirty days or more. In the exemplary embodiment, data is gathered and stored in memory in a round robin method, and will always contain the last thirty days of data. Any data older than thirty days will be over written and lost. An alternative memory may be used in which all data is stored on a long-term basis.

The control unit includes an appropriate memory and microprocessor for storing data in the memory. The memory can take any acceptable form such as DRAM, SRAM, EEPROM magnetic storage media, disk or the like. The microprocessor will collect such data as the water flow rate continuously or, over selected time periods. It will also collect and store the voltage provided to the plates and the actual current that passes through the plates for the given voltage. The microprocessor also provides a time correlation signal for each of the stored data components so that they may be correlated exactly with each other. For example, the data is stored in such a way that the readout from the memory provides a time correlation between the water flow rate and the current and voltage at a given time. For any given flow rate at a particular time, the current and voltage over the same time period can be known and reviewed. Since each of the values are stored on a time correlated basis, the response of the electronic controller to changes in the water flow rate can be precisely monitored as well as the amount of time required for the response to occur. In addition, the time correlation between a change in voltage and variations in current can also be monitored. According to one alternative embodiment, the output from the water softener may also be monitored and be time correlated with changes in the voltage and current density through the electrolytic cell.

According to one embodiment, the current and voltage values are converted into digital forms and stored as bytes that can be directly translated into the respective analog values. The electronic signal from the water flow meter is also stored as a digital byte but it can easily be transformed into an analog decimal value so as to determine the gallons per minute of the particular flow rate (or, as desired liters per minute depending on the conversion unit).

According to one embodiment, the microprocessor also stores the current and voltage as averaged over a particular six-hour period during which the cell is active. If the cell is not active at all for an entire six-hour period, the value as stored will be zero for both current and voltage. The average flow rate will be stored for its actual value during that period. Alternatively, if the cell is active for a portion of the six-hour period then, the average current and voltage over that six-hour period is stored. This embodiment has significant advantages in providing data compression for both storage and transmission. Each twenty-four hour day is broken into four, six-hour periods. The average current for each six-hour period is stored, as is the average voltage. Thus, in any given day there were eight data points stored for power, four for current and four for voltage. Data is also stored which provides the time correlation for the date and time of day for each of the respective four data points for current and voltage. The flow is also averaged and stored for the six-hour period. Thus, for each time period only four bytes need be stored, four times a day. A first byte providing the date and time of day, a subsequent byte providing the current, subsequent byte providing the voltage, and the final byte providing the average flow rate. These four bytes are then stored in the memory accessible by the microprocessor. The bytes can then read out transmitted for storage in a master computer, as explained herein. In one embodiment, to save even more memory, a date and time byte need not be stored with each time period. Instead, a starting date and time are known. The subsequent bytes are stored in the order they are collected and read out exactly in the order collected. Thus, the correct byte is sent, the voltage byte and the flow rate byte, followed by the next set of current, voltage and flow bytes. The master computer at the base location knows the starting date and time. It can thereafter add the date and time data and correlate it with the stored data at the master computer. In this embodiment, the date values are stored as raw data in a selected sequence without a limited time correlation as stored. This saves data storage space at remote site 32. Upon being read out, it can be presented to the user in sequence within the time and date. If needed, the master computer can add the date and time correlation in the software program stored at the master site. The data transmission will be rapid and reliable, using this 3-byte sequence for each time period.

In summary, the data can be stored using various alternative techniques, each of which has advantages. According to the first technique, the actual current and voltage are directly monitored and stored on a real-time basis, together with the time correlation signal. According to the alternative embodiment, the current, voltage and flow are determined for selected time periods and stored, together with an indication of the time period. This can be done four times a day, for six-hour time periods as has just been described. As a further alternative, the data may be stored and compressed using any other acceptable technique as will be appreciated as equivalent.

The electronic controller contains the communication connector 74 and also access ports 76. The communications connector 74 may be any acceptable connection to a computer system such as an RS232 connector, a modem, a high-speed universal bus connector or any other acceptable communications connector. According to a preferred embodiment, a main computer is connected to the electronic controller 32 via the communications connector on a regular basis, such as once a week, every thirty days, or some other periodic basis. The stored data is then transferred to a master base site that has large storage capability. The master base computer may also have a large display monitor and a video graphics package so as to display the data in various forms such as in tables, graphic layouts or other acceptable techniques that provide easy viewing for those persons monitoring the performance of the electrolytic cell. After the data is downloaded and stored at the master location, from the remote location the memory in the electronic controller 32 can be erased or, if desired, written over since it is no longer needed to be stored within the remote location at the controller 32 because it is stored and saved at the master location. Of course, the electronic controller 32 will also store whether the main power is turned on or off, as well as whether the check system signal has been generated to determine whether or not the system needs to be personally checked or have maintenance performed.

In the exemplary embodiment, the communication package includes a modem with modem speed of 4800 baud with a 4 MHz crystal. Alternatively, modem speed of 9600 baud would require an 8 MHz crystal. The active modem located in the controller panel allows a user to remotely call in and check or change parameters. Alternatively, a connection is located in the controller panel such that the user can hook up a laptop computer to perform the date measurements and make adjustments on site. In yet another embodiment, the amperage, voltage and flow rate can be adjusted manually on site at the controller panel. This flexibility allows the user to monitor and optimize the system at all times.

In operation, the treatment system 11 of the present invention combines to remove impurities that adversely effect water quality; prevents scaling and sediment contamination to the piping and drinker lines; and improves the quality of the water delivered to the animals. The water treatment filter initially removes impurities in the water to allow the water to be treated by the electrocatalytic cell. The electrocatalytic cell increases the dissolved oxygen in the water and further converts contaminants into inert particles or gases that can settle out of the system or be off-gassed. The electrocatalytic cell splits the water molecules into their component parts of hydrogen and oxygen. Other molecules will also be split, for example, chlorine compounds or molecules and hydrocarbons are also split apart. The hydrogen molecules are then allowed to bond with contaminants to form a precipitant while the oxygen is allowed to dissolve in the holding chamber. The sediment filter filters out sediment that may clog feeder lines prior to the treated water entering the livestock drinking system. Thus, the combination provides a cleaner, less contaminated, higher quality of water in an efficient and cost effective manner.

The treatment system 11 has multiple set points and is capable of a current adjustment sensitivity equal to approximately one-third gallons per minute variation in flow. In the field, after running the treatment system 11, evidence of twelve to sixteen PPM of dissolved oxygen has been achieved, showing significant benefit to the development of birds. Typically, levels in the field have been found in the range of six to nine PPM on average in chicken farms but have also been found as low as one PPM. Furthermore, the treatment system 11 will reduce the oxygen potential of water and may convert the water from an original oxidant level to an antioxidant, thus further benefiting the birds.

The examples below reflect that the increased levels of dissolved oxygen, which becomes dissolved in water as a result of the water treatment process described herein, is effected by the process that naturally dissolves some oxygen in all water. It is therefore present in the form of diatomic oxygen (O₂) and not as monatomic oxygen, ozone or peroxide. Therefore, the water remains completely natural.

Oxygen, in any of the forms mentioned above, is known to act as a disinfectant. Therefore by elevating the level of O₂ in water, the ability of the water to kill certain bacteria is increased. Experiments have shown that the passage of water through the equipment has the effect of sterilizing it. However this is not the primary purpose of the process. The water treatment process described herein makes good water better rather than making bad water good. Water that was used in the experiments described below was tap water, which was of good bacteriological quality to begin with (3-4 cfu/ml), but the process effectively sterilized it (0 cfu/ml).

Further experiments, in which oxygenized water was used as a mild disinfectant by adding it to media broth of selected bacteria, were conducted. It reduced the bacterial population by log 4(10,000 times less) when compared to tap water. The treated water was then fed to chickens in order to control the pathogenic bacteria of the chicken's intestinal tract.

When the gut microflora of a chicken is examined a very varied and diverse population is found. However in general it can be said that since it is an enclosed chamber nearly all the bacteria present are anaerobes (preferring the absence of oxygen to survive). Within the intestinal tract, two types of anaerobes can be identified, those that can survive in the presence of oxygen (facultative anaerobes), and those that cannot (strict anaerobes). The strict anaerobes tend to dominate in the chicken's gut. Within this group are the harmful organisms such as Clostridia, which are involved in causing the potentially fatal disease Necrotic Enteritis. Other pathogenic bacteria include for example, Salmonella, Shigella, Staphylococcus, Esherichi coli, and Helicobacter pylori. Currently these harmful bacteria are controlled by dietary inclusion of antibiotics, enzymes etc. However, with many countries in the world now banning antibiotic growth enhancers, control of Necrotic Enteritis or hindgut disease is more difficult. As illustrated in the experiments outlined in the examples, feeding birds treated water from the system described herein prevented the birds from developing Necrotic Enteritis.

Physiological experiments have shown that in the presence of the treated water, which includes elevated dissolved oxygen, the ratio of harmful bacteria, as represented by coliforms, compared to beneficial bacteria, the lactic acid producers, is altered in favor of the latter. Experiments confirm that the L group (beneficial bacteria) is increased rather than the C population (harmful bacteria) being reduced.

Oxygen and water are requirements of all living systems, and the amount of oxygen contained in water could have major implications for all forms of life. Water typically has a stabilized oxygen content of 4-6 ppm. The water treatment system of the present invention uses electro-catalytic chemistry to increase the stabilized oxygen content of water to the theoretical physical limit of 45 ppm. During this process described herein, the hydrogen and oxygen molecules within water dissociate, with the consequence that the level of dissolved oxygen within water is increased. The dissolved oxygen produced may be in the form of O₂⁻, O₂ or O₃.

All living cells are prone to oxygen toxicity. For some bacteria (strict anaerobes), often those associated with infections and disease, oxygen can be lethal. An oxygen molecule is short of one electron on its outer orbit and thus it will try to acquire an electron to replace the missing electron orbiting the oxygen molecule. When this oxygen molecule encounters an infectious or putrefying bacteria, it will strip the electron away from the outer protective membrane of the organism. Without the electron on its outer protective membrane, the anaerobic bacteria cannot survive. Conversely, there are anaerobic bacteria (facultative anaerobes) that live within the normal gut flora of animals that can withstand the presence of oxygen. Facultative anaerobes are beneficial to their host.

Oxygen therapy involves providing increased levels of dissolved oxygen to mix with gastric fluid throughout the digestive tract. According to principles of the present invention, the water treatment system provides water with increased dissolved oxygen. Ingesting the treated water provides increased oxygen to the gut may and results in a reduction in the total flora, which results in less energy being used to support intestinal flora and more energy being available for growth. Thus, improving feed conversion efficiency and weight gain of, for example, poultry. Treated water containing increased levels of dissolved oxygen, therefore, has huge potential in an industry such as the poultry industry which faces a ban on the use of sub-therapeutic antibiotics. In addition, increasing the oxygen level in the gut of broilers will reduce the level of pathogenic anaerobes, leading to a safer final product.

Further benefits to using dissolved oxygen in the poultry industry may include, reduced mortality, optimum functioning of the gastrointestinal immune response system and increased absorption of glucose, vitamins and essential minerals. An additional benefit of the present invention includes making medicine more effective and efficient both because of the improved health of the birds as well as when provided as mixed in the treated water. One possible explanation for why the treated water enhances the nature of the administered medicament is because oxygen is a highly reactive compound. Empirical results as shown in Example 1, from experimental testing, of the technology to supply drinking water to broiler chickens have shown significant beneficial effects on bird health and performance: specifically, reduced mortality, improved feed conversion ratios, increased liveweight, fewer factory downgrades, reduced use of antibiotics and more effective use of antibiotics, enzymes and the like resulting in a reduced required dose.

Increasing the molecular oxygen content in the intestine through providing the birds with treated water having a higher content of dissolved oxygen may alter the balance of flora in favor of the beneficial bacteria, thereby improving bird health and performance. Improvements in weight, feed efficiency and reduced mortality that have been observed under normal growing conditions, due, it is believed, to these changes to the gut microflora. Another possible explanation is that providing treated water with higher levels of dissolved oxygen increases the metabolism rate, thus providing beneficial results.

By reducing the numbers of strict anaerobes in the gut of the growing bird, the risk of infectious disease, and hence morbidity and mortality are reduced. This in turn allows the beneficial bacteria to proliferate thereby enhancing the digestion and absorption of available nutrients to the bird. Theoretically the net effect of this is likely to be greater body weight and improved feed efficiency, as has been observed.

The following example of a communication session is provided for illustrative purposes only.

### COMMUNICATION FORMAT FOR MODEM

The following is one example of a communication format to be used with the modem.

1. Modem speed is set at 4800 baud with a 4 MHz crystal.

2. Once connected, the calling program send the following string:

Read WC065

Followed by a carriage return.

3. The response data is as follows:

V### where ### is the firmware version number-all characters will be ASCII

T# where # is the cell reversal time-all characters will be ASCII

E# where # is a hex digit, bits 7-6 will be 0, bits 5-0 will be set only if an error condition exits. The error conditions are as follows:

bit 5-No AC on supply number 3

bit 4-No AC on supply number 2

bit 3-No AC on supply number 1

bit 2-over voltage error. System is shut down until serviced.

bit 1-over current error. System is shut down until serviced.

bit 0-under current error. System is shut down until serviced.

The remaining data will be the current, voltage and pump flow readings for the last 30 days. Current and voltage are averaged over a 6-hour period, and only while the cell is active. If the cell is not active for the entire 6-hour period the values will all be 0 for I and V. Flow will be stored as whatever the average flow was over that time period. Current and voltage values will be stored in Hex bytes that can be directly translated to their respective values. Pump flow will also be a Hex byte, and it can be translated to decimal and multiplied by 12 and then divided by 174 to determine the gallons per minute: (flow*12)/174=gpm.

4. The following pattern is repeated until all data is sent. (1080 bytes)

I# where # is a hex byte representing the average current over a 6-hour period.

V# where # is a hex byte representing the average voltage over a 6-hour period.

F# where # is a hex byte representing the average flow over a 6-hour period.

5. The data is finished when the following string is received: END

Total byte count is 1091 bytes or 8728 bits. Approximately 2 seconds of data @ 4800 baud. Once the data transmission is complete, the connection will be terminated immediately. Data is gathered in a round robin method.

### EXAMPLES

### Example 1

In each of three separate growth trials, 20,000 birds were grown to 40 days of age in houses either provided with oxygenized water or supplied with normal water. At 40 days of age, 20 birds were removed from each house and transported to the Queen's University of Belfast.

Birds were then sacrificed using cervical dislocation and their gut microflora studied using samples of proximal ileal and caecal digesta. Appropriate dilution series were prepared and digesta samples were plated onto MRS agar for determination of presumptive numbers of lactic acid bacteria and Maconkey agar for presumptive numbers of coliforms. The MRS:MAC ratio is used as an indication of the microfloral balance within a bird, a higher ratio indicating a more beneficial flora.

TABLE 1
MRS:
MAC ratios for digesta samples removed from the ileam and caecum

| | Control | Oxygenized |
|---|---|---|
| Replicate 1 | | |
| Ileum | 1.74 | 3.48 |
| Caecum | 0.55 | 5.42 |
| Replicate 2 | | |
| Ileum | 1.43 | 3.06 |
| Caecum | 1.04 | 3.32 |
| Replicate 3 | | |
| Ileum | 0.82 | 1.81 |
| Caecum | 0.44 | 1.80 |

In each of the three separate trials, the MRS:MAC ratio was markedly higher in both the ileum and caecum when birds were supplied with oxygenized water. This effect was mainly due to the numbers of lactic acid bacteria being increased in both the ileum and caecum when birds were provided with oxygenized water.

The research was conducted to investigate the chemistry, bacteriology and physiology of the use of oxygenated water for broiler chickens to try and establish the mechanisms at work with the longer-range goal of determining optimum conditions for the use of oxygenated water. The following examples provide specific data with respect to the research and testing.

### Example 2

### Determination of the Relationship between Dissolved Oxygen Concentration and Flowrate through the Cell

Aims and Objectives

To quantify the amount of dissolved oxygen produced in water that has passed through the water treatment equipment system described herein at varying water flow rates and levels of electric current.

Materials and Methods

The amperage at which the AHS-oxygenize operates shall be adjusted via the flow of water entering the system over a practical range (20 to 40 Amps). After passing through the water oxygenizer samples shall be taken and dissolved oxygen, total chlorine, free chlorine, pH and alkalinity measurements made.

TABLE 1
Effect of increasing flow rate and electrical current on dissolved
oxygen content and chemical profile of oxygenized water

| | | | | | |
|---|---|---|---|---|---|
| Amps | 20 | 25 | 30 | 35 | 40 |
| Dissolved Oxygen (ppm) | 14.4 | 13.3 | 13.5 | 12.9 | 12.5 |
| pH | 7.2 | 7.2 | 7.2 | 7.2 | 7.2 |
| Total alkalinity (ppm) | 40-80 | 40-80 | 40-80 | 40-80 | 40-80 |

Dissolved oxygen content of the water samples decreased with increasing flow rate through the water treatment equipment. Both pH and total alkalinity were unaffected by altering the flow rate of water passing through the electrolytic cell and consequently the electric current at which the equipment is operating.

Discussion and Conclusions

The results are shown graphically in FIG. 1 and this illustrates that the rate of decrease of dissolved oxygen with increasing flow rate is linear (R²=0.95). A compromise therefore has to be obtained in any situation between the amount of water required and achieving the maximum amount of dissolved oxygen. In a local field trial using the water treatment system, referred to as AHS-oxygenizer, in a broiler production house with a bird capacity of 20,000, during the first two weeks of life the oxygenizer is operating at approximately 20 Amps. Part of the current hypothesis as to the mode of action of the oxygenized water in animal husbandry systems is that it is affecting the gut bacterial population. As gut microflora are less stable during the early part of their hosts' life, preventing obligate anaerobic organisms from proliferating and therefore allowing beneficial facultative anaerobes such as the lactic acid bacteria to become established.

The other parameters measured were unaffected by flow rate and electrical current.

[image]

### Example 3

### Determination of the Molecular Form of Dissolved Oxygen Created

Aims and Objectives

To determine the molecular form of dissolved oxygen produced in water that has been subjected to the water treatment system described herein, used at constant temperature, flow rate and amperage.

Materials and Methods

Chemical forms of oxygen were determined in water samples produced using the AHS-oxygenizer at different flow rates.

Results and Discussion

Analyses of the water samples for the presence of nascent oxygen and ozone revealed that all oxygen produced using the water treatment system, referred to herein as the AHS-oxygenizer, is in the di-atomic form (O₂). Oxygen free radicals have a very short half-life and so nascent oxygen may be formed in the cell, but it certainly does not survive and is not detectable in water leaving the oxygenizer.

It is beneficial that dissolved oxygen produced by the equipment is in the form of O₂, especially if water which has been treated using the oxygenizer is to have application in human nutrition, as this is the most natural way in which it can appear.

### Example 4

### Determination of the Lag Time Required for Optimum Dissolution of Oxygen In Water

Aims and Objectives

The aim of the current experiment is to determine the lag time required between water passing through the water ‘oxygenize’ at varying temperatures and its application.

Materials and Methods

Using a standard flow rate (1.5 l/min) and amperage (20.3 Amps), at 5° C., oxygen stability of a sample of water discharged from the AHS-oxygenizer shall be monitored with time over the first 10 minutes post processing. Dissolved oxygen shall be measured using a dissolved oxygen meter, stirring the water sample continuously with the probe during the observation period. This shall then be repeated at 10° C. and 20° C.

TABLE 2
Dissolved oxygen profile with time (mean of 4 observations)

| | | | | | | | | |
|---|---|---|---|---|---|---|---|---|
| Time (mins) | 0 | 1 | 2 | 3 | 4 | 5 | 6 | 7 |
| DO (ppm) | 9.9 | 14.5 | 16.2 | 16.6 | 16.7 | 16.7 | 16.7 | 16.7 |
| Time (mins) | 8 | 9 | 10 | 11 | 12 | 13 | 14 | 15 |
| DO (ppm) | 16.6 | 16.5 | 16.5 | 16.3 | 16.1 | 15.9 | 15.8 | 15.7 |

Dissolved oxygen content of the water sample was observed to increase steadily over the first 3 minutes post processing using the AHS-oxygenizer, where it then stabilized at a peak dissolved oxygen content of 16.7 ppm for a period of 4 minutes. During the remaining 7 minutes of the observation period, there was a slow, but steady decline in the dissolved oxygen content of the water sample. The results are illustrated graphically below in Graph 2 titled “Mean dissolved oxygen profiled over time.”

Discussion and Conclusions

The results shown in Table 2, indicate that a period of 3 to 4 minutes is required for dissolved oxygen to stabilize in water after treatment with an AHS-oxygenizer. In a commercial poultry house situation, where water passes from the oxygenizer to a water header tank, this will not pose a problem, as the residence time in a header tank is unlikely to be less than 4 minutes. If the oxygenized water is to be used in, for example, a food processing plant, it may be necessary to situate the equipment at a location that allows a period of 3-4 minutes to pass prior to its use for the most effective application.

The depletion of dissolved oxygen observed in the sample after 7 minutes of measurements taking place is due to the method of analysis and not due to instability of the dissolved oxygen present in the water. The instrument used for measuring dissolved oxygen comprises a membrane electrode composed of a cathode and anode in contact with an electrolyte solution. The measurement of oxygen is accomplished by applying a voltage across the sensor, reducing the oxygen and thus effectively removing it. As this type of system consumes oxygen, the amount detected in the water sample shall decrease with time.

[image]

### Example 5

### Bacteriology

### Determination of the Effect on the Bacterial Population of Contaminated Water Passed Through the Oxygenizer

Aims and Objectives

This experiment aims to determine the effect of passing contaminated water from a natural source through the water ‘oxygenize’ on its bacterial population. The levels of both aerobic and anaerobic organisms in the oxygenized water shall be measured.

Materials and Methods

Water samples shall be taken both pre and post-treatment with the water treatment equipment described herein and passed through a filter and compared with Ringers Solution for bacterial contamination.

Results and Discussion

The water sample that was examined pre-treatment with the water treatment equipment, referred to herein as AHS-oxygenizer, had a low level of bacterial contamination (3-4 cfu/100 ml). Oxygenized water samples taken post-treatment did not contain any colony forming units and so were effectively sterilized.

### Example 6

### Determination of the Effect of Oxygenized Water on the Growth of Anaerobic Micro-Organisms

Aims and Objectives

This experiment was conducted to determine the bactericidal effect of oxygenized water on a coliform bacterium in vitro.

Materials and Methods

Water samples were taken in a sterile manner both pre- and post-treatment with the water treatment equipment of the present invention. A coliform colony was inoculated into 60 ml of buffered peptone water and allowed to culture for 24 hours at 37° C. A colony count of the 24 hour undiluted culture was then determined (reported as bacterial numbers at t=0). The resulting culture was then diluted into Ringers Solution, containing the water samples taken pre- and post-treatment with the water treatment equipment of the present invention in 10 fold serial dilutions from 10⁻¹ to 10⁻⁶. Diluted cultures were then held at room temperature for 1 hour prior to plating. Samples from each dilution were plated onto TSA and incubated for 24 hours at 37° C. after which period the colonies were counted.

Results and Discussion

The results in Table 3 below are presented as the number of colony forming units (cfu) per ml of solution present after incubation with the water samples at room temperature for 1 hour. Column 2 in the table presents the number of coliforms present at time zero, prior to dilution using the water samples to illustrate the bactericidal potential of each sample. The results clearly demonstrate that the sample of oxygenized water is effective in reducing a culture of coliform bacteria at a concentration of 6.0×10⁴ cfu/ml to zero after incubation at room temperature for 1 hour. This is compared with the control sample of water taken pre-treatment using the water treatment equipment that still had an uncountable number of cfu/ml at this and indeed the next dilution in the series. The control water sample did not reduce the number of coliform bacteria present to zero at any point in the dilution series.

These results clearly demonstrate that oxygenized water has a powerful bactericidal effect against facultatively anaerobic coliform bacteria.

TABLE 3
Reduction in mean numbers of bacteria
present in a culture of coliform bacteria after
incubation at room temperature with control and oxygenized water

| | Bacterial numbers | | |
|---|---|---|---|
| Dilution | present at t = 0 | Control | Oxygenized Water |
| 1 × 10⁻¹ | 6.0 × 10⁶ | UC | UC |
| 1 × 10⁻² | 6.0 × 10⁵ | UC | 58 |
| 1 × 10⁻³ | 6.0 × 10⁴ | UC | 0 |
| 1 × 10⁻⁴ | 6.0 × 10³ | UC | 0 |
| 1 × 10⁻⁵ | 6.0 × 10² | 89 | 0 |
| 1 × 10⁻⁶ | 6.0 × 10¹ | 30 | 0 |

Physiology

The study of the relationship between oxygenized water and bird physiology is a work in progress with several components. Reported below are the results of the work completed to date.

### Example 7

### Determination of the Effect of Oxygenized Water on Performance, Carcass Characteristics and Gut Microflora of Broiler Chickens

Aims and Objectives

To examine the effect of using oxygenized drinking water on the performance, carcass characteristics and gut microflora of broiler chickens.

Materials and Methods 40 male broiler chicks shall be obtained at day-old and kept in a brooder at an initial temperature of 33° C. for one week. At 7 days of age, birds shall be weighed and randomly allocated to one of two groups, those being given normal water and those being given oxygenized water. Birds shall then be placed in individual metabolism cages. Birds shall be fed ad libitum on a commercial diet and feed and water intake measured on a weekly basis from 7 to 35 days of age. Bids shall be weighed at the end of each week for calculation of feed conversion efficiency.

Birds shall be slaughtered at 35 days by cervical dislocation and the intestinal contents removed for microbiological examination. Carcasses shall be dissected for the measurement of breast meat yield and fat deposition.

Results and Discussion

Ileal and caecal samples were removed from 35-day-old broilers that had been reared at a commercial trial facility with and without access to oxygenized water. The intestinal samples were analysed using both MRS agar and MAC broth to determine the numbers of lactic acid bacteria and coliforms respectively. The MRS:MAC ratio is used as an indication of the microfloral balance within a bird, a higher ratio indicating a more beneficial flora.

The MRS:MAC ratio determined using ileal samples from birds grown using oxygenized water was twice that of the control birds. The effect of oxygenized water on the MRS:MAC ratio from the caecal samples was even more marked. The ratio in both cases was increased mainly through promotion of the lactic acid bacteria, rather than reduction in the numbers of coliforms present.

The results of these experiments reported in the examples illustrate that the use of oxygenized water can improve bird health and performance through promotion of the beneficial lactic acid producing bacteria within the birds gastrointestinal tract.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of increasing the lactic acid producing bacteria in the intestine by ingesting treated water wherein the treated water contains elevated levels of dissolved oxygen.

2. The method of claim 1 wherein the levels of dissolved oxygen in the treated water are greater than 8 parts per million (ppm).

3. The method of claim 1 wherein the levels of dissolved oxygen in the treated water is in the range of 10-45 parts per million (ppm).

4. A method of decreasing the coliform producing bacteria in the intestine by ingesting treated water wherein the treated water contains elevated levels of dissolved oxygen.

5. The method of claim 4 wherein the levels of dissolved oxygen in the treated water are greater than 8 parts per million (ppm).

6. The method of claim 4 wherein the levels of dissolved oxygen in the treated water is in the range of 10-45 parts per million (ppm).

7. A method of reducing the bacterial load in the digestive tract of poultry by feeding the poultry treated water containing elevated levels of dissolved oxygen.

8. A method of preventing necrotic enteritis in poultry by feeding the poultry treated water containing elevated levels of dissolved oxygen.

9. A method of treating water comprising,
inputting water to a water treatment filter;
modifying the water in the water treatment filter to remove minerals that are present in hard water;
outputting the water from the water treatment filter;
measuring the flow rate of water after it has left the water treatment filter;
inputting the water to an electrocatalytic converter which causes electrical current to pass through the water;
flowing the water out of the electrocatalytic converter into a holding chamber;
pressurizing the holding chamber; and
outputting the water from the holding chamber into a pipeline connected to a poultry drinking system.

10. The method according to claim 9 wherein the step of modifying the water includes removing selected metals from the water.

11. The method according to claim 9, further including replacing the calcium atoms removed from the water with sodium to make the water soft water.

12. The method according to claim 10 wherein the calcium that is removed includes magnesium and the replacement atom includes sodium.

13. The method according to claim 9, further including outputting a measurement of the flow rate into an electronic sensor, and modifying the current density flowing through the water from the electrocatalytic converter based on the value of the flow rate.

14. The method according to claim 13 wherein the amount of current increases proportional to increases in the flow rate.

15. The method of claim 9 wherein the pressurizing of the holding chamber includes holding the treated water in a pressure tank for a specified amount of time to allow further dissolution of oxygen into the water.

16. An apparatus for treating water comprising:
an electrocatalytic cell having a plurality of conductive plates with spaces therebetween through which water may pass;
an inlet for water to flow into the electrocatalytic cell;
a flow meter coupled in the flow line prior to the inlet for measuring the flow rate of water into the inlet;
an outlet from the electrocatalytic cell for receiving water which has passed through the electrocatalytic cell;
a pressure tank coupled to the outlet for receiving and pressurizing the treated water; and
an outlet pipe coupled to an outlet from the pressure tank for supplying the treated water for an end use.

17. The apparatus according to claim 16, further including a gas release valve coupled to the outlet for permitting gas to escape in the outlet prior to the outlet pipe.

18. The apparatus according to claim 16, further including a water treatment filter positioned in the water inlet line prior to the flow meter for modifying the hardness of the water prior to water flowing into the electrocatalytic cell.

19. The apparatus according to claim 16, further including a control unit to modify the current density in the conductive plates based on the flow rate of the water.

20. The apparatus according to claim 19 wherein the control unit further includes a memory storage unit coupled to a control unit, the memory storage unit storing an on/off time, a value of the current flow, and a flow rate on a continuous real-time basis.
